(19) United States
(12) Patent Application Publication
(10) Pub. No.: US 2004/0214786 A1
(43) Pub. Date: Oct. 28, 2004

(54) Heterostructure component

(76) Inventors: Franz Hofmann, Munchen (DE); Richard Johannes, Munchen (DE); Thomas Schulz, Munchen (DE); Wolfgang Rosner, Ottobrunn (DE)

Correspondence Address: ALTERA LAW GROUP, LLC, 6500 CITY WEST PARKWAY, SUITE 100, MINNEAPOLIS, MN, 55344-7704, US

(21) Appl. No.: 10/474,606

(22) PCT Filed: Apr. 11, 2002
(86) PCT No.: PCT/DE02/01362

(30) Foreign Application Priority Data
Apr. 12, 2001 (DE) 101184050

(51) Int. Cl. A61K 48/00; C07H 21/04

(52) U.S. Cl. 514/44; 536/23.1; 423/445 B

(57) ABSTRACT

The invention provides a very compact, yet reliable heterostructure and method of manufacture thereof. The invention provides a heterostructure component, and method of manufacture, having a single hetero-nanotube, which includes: a first region made from a first nanotube material with a first value for the bandgap, and a second region made from a second nanotube material having a second value for the bandgap, which is different from the first value for the bandgap. The second region is arranged at the upper end of the first region in the longitudinal direction of the hetero-nanotube. The first nanotube material is a different material than the second nanotube material.

### DESCRIPTION

The invention relates to a heterostructure component.

Nowadays, electronic components are fabricated predominantly on the basis of silicon MOS structures (MOS=metal oxide semiconductor) or semiconductor heterostructures.

A typical simple silicon MOS structure is composed of a layer structure having a semiconductor silicon layer, an oxide layer (SiO₂) formed on the silicon layer and a metal layer formed on the oxide layer. If a sufficiently high positive electric field is applied to the metal layer, a conductive channel region is formed as a result of a field effect in a region of the silicon layer which adjoins the oxide layer. The level of conductivity of the channel region can be altered by means of the field strength of the applied electric field.

A typical semiconductor heterostructure is composed of at least two different compound semiconductor materials which are arranged in layers on top of one another and have different bandgaps between valence band and conduction band but the lattice constants of which differ only slightly from one another. On account of the fact that their lattice constants differ only slightly, the two different materials can be grown on top of one another without any dislocations, so that a heterogenous crystal with two layers each comprising a different compound semiconductor material is produced, yet the lattice constant is the same throughout the entire heterogenous crystal. If the difference in the bandgap of the two different compound semiconductor materials is suitable, a potential minimum is formed at the interface between the two different compound semiconductor materials. Dopants have been introduced into at least one of the compound semiconductor materials. The dopants provide charge carriers which can move approximately freely in the heterogenous crystal and accumulate in the potential minimum, so that a conductive layer is formed at the interface. Heterostructures without dopants are also produced, in particular for optical applications.

An example of a pair of compound semiconductor materials which is particularly suitable for the production of a heterostructure is the two compound semiconductor materials gallium arsenide (GaAs) and aluminum gallium arsenide (AlGaAs).

A further pair of compound semiconductor materials which is suitable for production of a heterostructure is silicon/silicon germanium (Si/SiGe).

Further typical suitable combinations of materials for semiconductor heterostructures are InP/InGaAsP and InP/InGaAlAs.

Given a suitable choice of the order of the different layers, it is possible for different electronic and opto-electronic components, such as for example diodes, transistors and lasers, to be realized from layer structures having a plurality of layers each with a different bandgap arranged on top of one another.

Conventional silicon MOS and compound semiconductor heterostructure techniques are approaching their limits with ongoing miniaturization.

Carbon nanotubes are known as semiconducting and metallically conducting structures of very small dimensions, cf. for example [1]. Carbon nanotubes are fullerenes formed from carbon atoms which are arranged so as to form a tube-like crystalline structure. They can be produced with a diameter of 0.2 nanometer up to approx. 50 nanometers and above and a length of up to several micrometers. The diameter is typically 2 to 30 nm, and the length up to a few hundred nanometers. The bandgap for conduction electrons and therefore the electrical conductivity of the carbon nanotube can be adjusted by means of its tube parameters, such as for example its diameter and its chirality.

However, nanotubes can be produced not only from carbon but also from boron nitride; the latter are similar to carbon nanotubes and are known to be lattice-compatible with carbon nanotubes, i.e. the same crystal structures are available to them for crystallization as are available to carbon nanotubes (cf. [2]). Boron nitride nanotubes always have an insulating electrical conductivity characteristic, irrespective of the tube parameters such as diameter or chirality of the boron nitride nanotube, with the electronic bandgap being 4 eV (cf. [3]).

Known processes for producing nanotubes are vapor phase epitaxy (CVD=chemical vapor deposition), the arc discharge technique and laser ablation.

[4] discloses a process which allows a carbon nanotube to be converted into a boron nitride nanotube by means of a chemical substitution reaction. In this case, a hot atmosphere comprising gaseous boron and nitrogen is generated in an area surrounding the carbon nanotube which is to be converted. If the temperature of the atmosphere is high enough, a chemical substitution reaction occurs, in which carbon atoms in the carbon nanotube are replaced by boron atoms and nitrogen atoms.

[5] describes a carbon nanotube which has two regions with different bandgaps.

A similar carbon nanotube is proposed in [6].

[7] describes a process for producing nanotubes using catalyst material.

Furthermore, [8] describes a multi-walled nanotube having an inner structure comprising carbon layers, a middle structure comprising boron nitride layers and an outer structure comprising carbon layers.

It is an object of the invention to provide a very compact yet reliable heterostructure component.

The object of the invention is achieved by a heterostructure component as described in the independent claim.

The invention provides a heterostructure component having a single hetero-nanotube, which includes: a first region made from a first nanotube material with a first value for the bandgap, and a second region made from a second nanotube material having a second value for the bandgap, which is different from the first value for the bandgap. The second region is arranged at the upper end of the first region in the longitudinal direction of the hetero-nanotube. The first nanotube material is a different material than the second nanotube material.

The heterostructure component is designed in the form of a single hetero-nanotube with two regions (sections in the longitudinal direction of the hetero-nanotube) each having a different bandgap. In this context, the term “hetero-nanotube” means that the nanotube is heterogenous, in the sense that it has at least two regions in which the nanotube has in each case a different electronic bandgap. The term “hetero-nanotube”, analogously to the term semiconductor eterostructure, means that the nanotube is formed from two or more semiconductors with different bandgaps.

The hetero-nanotube may have more than two regions, i.e. for example may have a further region made from a material with a further value for the bandgap, which differs at least from the first value for the bandgap or from the second value for the bandgap. The further region is arranged at the upper end of the second region in the longitudinal direction of the hetero-nanotube. In this case, the hetero-nanotube may, for example, have the general structure “1-2-3” or the general structure “1-2-1” in its longitudinal direction, where 1, 2 and 3 symbolize three different bandgaps.

The value of the bandgap in the first, second and further regions may in particular in each case correspond to a conductivity characteristic from the group consisting of metallically conducting, semiconducting and insulating conductivity characteristics.

For example, a hetero-nanotube with two different regions may, for example, be configured in such a way that the hetero-nanotube is insulating in the first region and metallically conducting in the second region. Alternatively, the hetero-nanotube may be semiconducting in the first region and insulating or metallically conducting in the second region. Alternatively, the hetero-nanotube may be semiconducting in both regions, but with the bandgap in the first region differing from the bandgap in the second region. In very general terms, the bandgap in the first region is different than the bandgap in the second region, although the conductivity characteristic does not necessarily have to be different.

A purely semiconducting hetero-nanotube with three regions may, for example, be semiconducting with a first bandgap in the first region, semiconducting with a second bandgap, which is different than the first bandgap, in the second region and semiconducting, either with a third bandgap which is different than the first bandgap and the second bandgap or with the first bandgap, in the third region.

In a region in which the hetero-nanotube is metallically conducting, the hetero-nanotube may be formed as a metallically conducting carbon nanotube.

In a region in which the hetero-nanotube is semiconducting, the hetero-nanotube may be formed as a semiconducting carbon nanotube.

In a region in which the hetero-nanotube is insulating, the hetero-nanotube may be formed as a boron nitride nanotube.

The heterostructure component is extremely compact, with a diameter of 0.2 nm to 50 nm, typically 0.7 nm to 40 nm, and a length of 10 nm to 10 μm, typically 20 nm to 300 nm. On account of the good controllability with which carbon nanotubes and boron nitride nanotubes can be produced and with which the conductivity properties of a carbon nanotube can be set, the heterostructure component can also be produced with a high controllability and with desired properties.

Exemplary embodiments of the invention are illustrated in the figures and explained in more detail below. In the figures:

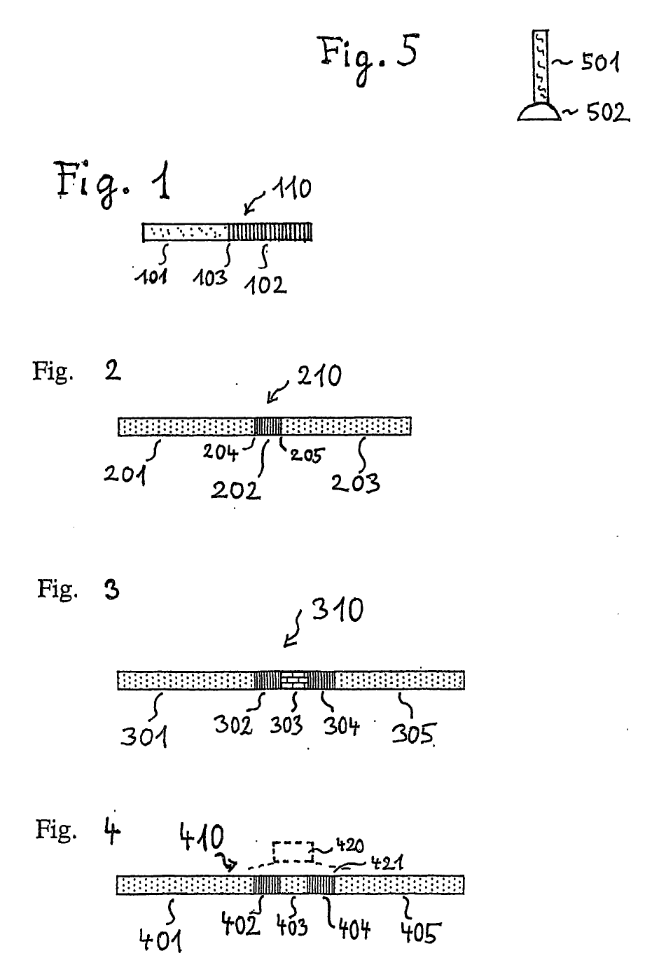

FIG. 1 shows a heterostructure component in accordance with a first embodiment of the invention;

FIG. 2 shows a heterostructure component in accordance with a second embodiment of the invention;

FIG. 3 shows a heterostructure component in accordance with a third embodiment of the invention;

FIG. 4 shows a heterostructure component in accordance with a fourth embodiment of the invention; and

FIG. 5 shows a nanotube arranged on a catalyst surface, in accordance with a variant of the invention.

The illustrations in the figures are diagrammatic and not to scale.

FIG. 1 shows a heterostructure component in accordance with a first embodiment of 2 5 the invention. The heterostructure component is designed in the form of a single hetero-nanotube 110 with an overall length of 400 nm and a diameter of 20 nm. The hetero-nanotube 110 includes a first region 101, which is formed from a metallically conducting nanotube, and a second region 102, which adjoins the first region and is formed from an electrically insulating boron nitride nanotube. The first and second nanotubes are arranged so as to adjoin one another in the longitudinal direction of the hetero-nanotube (110), so that the longitudinal axes of the first nanotube, the second nanotube and the overall hetero-nanotube 110 which is formed coincide, i.e. run parallel to one another on a single straight line. The first nanotube, which extends in the first region 101, ends at the upper end 103 of the first region 101, and the second nanotube, which extends in the second region 102, starts at the upper end 103 of the first region 101. The first region 101 and the second region 102 each have a length of 200 nm.

On a scale in the region of the distance between adjacent atoms in the nanotube, the carbon nanotube and the boron nitride nanotube may engage with one another a little at the upper end 103, this engagement being caused by the discrete crystalline structure of the nanotubes.

It is preferable for the first nanotube and the second nanotube to be formed in the same crystal structure. The first and second nanotubes may have an identical or different chirality. The chirality cannot be selected completely arbitrarily, but rather should be selected in such a way that the corresponding nanotube has the desired bandgap and/or the desired conductivity characteristic. It is also preferable for the first nanotube and the second nanotube to be fitted together as far as possible without dislocations. Realistically, however, it is possible that the hetero-nanotube will have dislocations in an annular region at the upper end 103 and around the upper end 103. These dislocations generally alter, usually adversely, the conductivity of the hetero-nanotube in the annular region.

In a further embodiment of the invention (not shown), which with the exception of the dimensions of the hetero-nanotube corresponds to the embodiment shown in FIG. 1, the diameter of the hetero-nanotube is 2 nm, its total length is 30 nm and the length of the first and second hetero-nanotube regions is in each case 15 nm.

In a further embodiment of the invention (not shown), which with the exception of the dimensions of the hetero-nanotube corresponds to the embodiment shown in FIG. 1, the diameter of the hetero-nanotube is 40 nm, its total length is 500 nm, the length of the first hetero-nanotube region is 200 nm and the length of the second hetero-nanotube region is 300 nm.

FIG. 2 shows a heterostructure component in accordance with a second embodiment of the invention. The heterostructure component has a hetero-nanotube 210 with a total length of 100 nm and a diameter of 1 nm.

The hetero-nanotube 210 includes a first region 201, which is formed from a metallically conducting nanotube, and a second region 202, which adjoins the first region 201 and is formed from an electrically insulating boron nitride nanotube. The first region 201 and the second region 202 are arranged in a corresponding way to the first region 101 and the second region 102 of the hetero-nanotube 110 shown in FIG. 1. Compared to the hetero-nanotube 110 shown in FIG. 1, the hetero-nanotube 210 additionally includes a further region 203, which is formed from a metallically conducting third carbon nanotube. The third nanotube is arranged at the upper end of the second nanotube in the same way as the second nanotube is arranged at the upper end of the first nanotube, i.e. the respective longitudinal axes of the first nanotube, the second nanotube, the third nanotube and the overall hetero-nanotube 110 which is formed coincide, i.e. run on a single straight line. The first nanotube, which extends in the first region 201, ends at the upper end 204 of the first region 201, and the second nanotube, which extends in the second region 202, begins at the upper end 204 of the first region 201. The second nanotube, which extends in the second region 202, ends at the upper end 205 of the second region 202, and the third nanotube, which extends in the further region 203, begins at the upper end 205 of the second region 202. The first nanotube and the second nanotube are therefore fitted together at the upper end 103 of the first region 101. The first and third regions 201, 210 each have a length of 49 nm in the longitudinal direction of the hetero-nanotube 210. The second region 202 has a length of 2 nm and forms a thin boron nitride ring which is embedded between two metallic carbon nanotubes.

The heterostructure component illustrated in FIG. 2 is in the functional form of a simple tunnel junction, the insulating boron nitride nanotube in the second region 200 serving as a tunneling barrier between the conductive first nanotube in the first region 201 and the conductive third nanotube 210 in the further region.

The above considerations relating to engagement between adjacent nanotubes in the longitudinal direction and dislocations in the junction region close to the boundary between two adjacent nanotubes in the longitudinal direction likewise apply to any hetero-nanotube with more than two regions, i.e. for example to the hetero-nanotube 210 shown in FIG. 2 and also the hetero-nanotubes 310, 410 which are described below and are shown in FIG. 3 and 4, respectively.

FIG. 3 shows a heterostructure component in accordance with a third embodiment of the invention. The heterostructure component includes a hetero-nanotube 310 with an overall length of 160 nm and a diameter of 0.8 nm. In terms of its structure, the hetero-nanotube 310 resembles the hetero-nanotube 210 shown in FIG. 2, with the main difference being that two insulating boron nitride nanotubes 302, 305 and a semiconducting carbon nanotube 304 embedded between the two boron nitride nanotubes 302, 305 are provided instead of the insulating boron nitride nanotube in the second region 202. Overall, therefore, the hetero-nanotube 310 includes, as seen from the left to the right in FIG. 3: a first region 301 with a length of 70 nm, which is formed from a metallically conducting carbon nanotube; a second region 302 with a length of 2 nm, which is formed from an insulating boron nitride nanotube; a third region 303 with a length of 3 nm, which is formed from a semiconducting carbon nanotube; a fourth region 304 with a length of 2 nm, which is formed from an insulating boron nitride nanotube; and a fifth region 305 with a length of 83 nm, which is formed from a metallically conducting carbon nanotube.

The heterostructure component illustrated in FIG. 3 is in the functional form of a resonant tunneling diode having an insulator-semiconductor-insulator layer sequence which is formed by the regions 302-303-304 and is embedded between a “left-hand” (in the illustration shown in the figure) conductive layer formed in the first region 301 and a “right-hand” conductive layer formed in the fifth region 305.

The resonant tunneling diode can be used, for example, in high-frequency electronics or as a module for an alternative logic to field-effect transistor logic in which field-effect transistors are used to realize logic circuits.

FIG. 4 shows a heterostructure component in accordance with a fourth embodiment of the invention. The heterostructure component includes a hetero-nanotube 410 with an overall length of 210 nm and a diameter of 2.2 nm. In terms of its structure, the hetero-nanotube 410 resembles the hetero-nanotube 310 shown in FIG. 3, the main difference being that a metallically conducting carbon nanotube is provided in the third region 403 instead of the semiconducting carbon nanotube in the third region 303. Overall, therefore, the hetero-nanotube 410 includes, as seen from the left to the right in FIG. 4: a first region 401 with a length of 113 nm, which is formed from a metallically conducting carbon nanotube; a second region 402 with a length of 1.5 nm, which is formed from an insulating boron nitride nanotube; a third region 403 with a length of 4 nm, which is formed from a metallically conducting carbon nanotube; a fourth region 404 with a length of 1.5 nm, which is formed from an insulating boron nitride nanotube; and a fifth region 405 with a length of 90 nm, which is formed from a metallically conducting carbon nanotube.

The heterostructure component illustrated in FIG. 4 takes the functional form of a single-electron tunneling diode with an insulator-conductor-insulator layer sequence which is formed by the regions 402-403-404 and is embedded between a “left-hand” conductive layer formed in the first region 401 and a “right-hand” conductive layer formed in the fifth region 405. In the third region 403, electrons can be stored by means of Coulomb blockade, the boron nitride nanotube in the second region 402 and the boron nitride nanotube in the fourth region 404 in each case serving as a tunneling barrier.

The single-electron tunneling diode shown in FIG. 4 can be used in combination with an additional gate electrode 420 as a single electron transistor. The additional gate electrode 420 extends next to the hetero-nanotube 410 and is arranged in such a way that an electric field can be applied to the fourth region 403, so that the energy levels for electrons in the third region 403 can be varied by means of this gate electrode 420, so that the Coulomb blockade can be produced or eliminated as a function of the voltage applied between the gate electrode 420 and the third region 403. An insulator layer 421 made from an insulating material, e.g. an oxide or a nitride, is provided between the gate electrode 420 and the hetero-nanotube 410.

Further elements may be provided at each of the heterostructure components illustrated in FIG. 1 to 4. By way of example, it is possible to provide conductive elements, by means of which the hetero-nanotube (110, 103) can be electrically connected to driving electronics. These conductive elements may, for example, be formed from metallically conductive carbon nanotubes, from metal, from doped polysilicon or from any other suitable conductive material. By way of example, metal may be vapor-deposited or sputtered onto one end of the nanotube. Alternatively, it is also possible for metal to be vapor-deposited or sputtered onto both ends of the nanotube. An electrical supply conductor may be electrically coupled to the conductive element and is also electrically coupled to the driving electronics, so that the hetero-nanotube and the driving electronics are electrically coupled. By way of example, a vapor-deposited metal strip or a further nanotube can be used as the electrical supply conductor.

The text which follows will explain a number of embodiments of a method according to the invention for producing a heterostructure component formed from a hetero-nanotube 110, 210, 310, 410.

In a first embodiment of the method, first of all a first nanotube is produced in a first region 101, 201, 202, and then a second nanotube is produced in a second region 102, 202, 203, fitting onto the upper end 103, 204, 205 of the first nanotube in the longitudinal direction of the first nanotube, so that overall a single hetero-nanotube 110, 210, 310, 410 is formed from the first nanotube and the second nanotube.

The nanotubes may in this case be produced, for example, by means of vapor phase epitaxy. In this case, first of all the first nanotube is produced on a base in a first vapor phase epitaxy step. Then, a second vapor phase epitaxy step is carried out, in which the second nanotube is produced on the upper end of the first nanotube. In the second vapor phase epitaxy step, the process conditions, such as process temperature, process pressure and process duration, are selected in such a way that in the second vapor phase epitaxy step the second nanotube is produced only on the first nanotube, by using selective epitaxy, whereas no further nanotubes are formed on the base. Alternatively, the nanotubes can be produced by means of an arc discharge technique or by means of laser ablation.

The method in accordance with the first embodiment can also be used to produce hetero-nanotubes with more than two regions with different nanotubes, such as for example the hetero-nanotubes 210, 310, 410 shown in FIG. 2, 3 and 4, respectively.

In a second embodiment of the method for producing a heterostructure component formed from a hetero-nanotube 110, 210, 310, 410, first of all a first nanotube is produced, then a second nanotube is produced, and then the second nanotube, fitted to the upper end 103, 204, 205 of the first nanotube in the longitudinal direction of the first nanotube, is attached to the first nanotube, so that a single hetero-nanotube 110, 210, which in a first region 101, 201, 202 comprises the first nanotube and in a second region 102, 202, 203 comprises the second nanotube, is formed from the first nanotube and the second nanotube.

In this second embodiment of the method, therefore, first of all individual nanotubes which are not connected to one another are produced, and these nanotubes are then joined together. By way of example, a suitable nano-manipulator, i.e. for example nano-forceps or a nano-suction-pipette or an electrostatically functioning nano-holding tool for electrostatically holding nano-particles or a similar tool, can be used to attach the second nanotube to the first nanotube.

It is optionally possible for in each case two nanotubes, after they have been assembled, to be welded together at the contact point at which they are in contact with one another, so that a reliable connection is produced between the two assembled nanotubes and a stable single hetero-nanotube is formed. The welding can be carried out, for example, by means of a local electric field which is applied to the two nanotubes in a predetermined region at the location of contact. A mask which shapes an electric field, which differs significantly from zero only in the predetermined region, can be used to generate the local electric field. Alternatively, the local electric field used may be the electric field beneath a fine conductive tip, for example beneath the tip of a scanning probe microscope.

The welding can be carried out, for example, by applying the local electric field as a short pulse. Alternatively, a constant electric field is applied for a longer period of time.

In the second embodiment of the method too, it is possible to use vapor phase epitaxy, an arc discharge technique or laser ablation to produce the first nanotube and/or the second nanotube and/or further nanotubes.

In a third embodiment of the method for producing a heterostructure component formed from a hetero-nanotube 110, 210, 310, 410, first of all a carbon nanotube is produced. Then, the carbon nanotube is converted into a boron nitride nanotube in at least a second partial section. For the hetero-nanotube 210 shown in FIG. 2, for example, first of all a carbon nanotube is produced by means of a conventional technique. Then, the carbon nanotube is converted into a boron nitride nanotube in the second region 202. The nanotube remains as a carbon nanotube in the first region 201 and in the third region 203. This creates the hetero-nanotube 210 illustrated in FIG. 2.

The carbon nanotube can be converted into a boron nitride nanotube as a result of a chemical substitution reaction being carried out.

The chemical substitution reaction can be effected by exposing the carbon nanotube which is to be converted to a sufficiently hot atmosphere containing boron atoms and nitrogen atoms until the chemical substitution reaction occurs. The atmosphere may, for example, be generated in a closed closeable chamber of a furnace which is suitably heated.

To ensure that the carbon nanotube is only converted into a boron nitride nanotube in a predetermined partial section, for example in the first partial section, it is possible, when carrying out the chemical substitution reaction, for the first partial section to be masked in such a way that it is shielded from the chemical substitution reaction, so that the chemical substitution reaction takes place only in the second partial section.

In this case, the chemical substitution reaction is carried out using a method which is based on the method for converting a carbon nanotube into a boron nitride nanotube which is known from [4] and was referred to in the introduction to the description. Compared to the method disclosed in [4], the method has been further developed by virtue of a suitable mask being used when the method is being carried out, so that only a partial region or only individual partial regions of the carbon nanotube are exposed to the atmosphere containing boron atoms and nitrogen atoms, and consequently the carbon nanotube is only converted into a boron nitride nanotube in these partial regions. To produce the hetero-nanotube 210 from FIG. 2, by way of example first of all a carbon nanotube is produced. The first region 201 and the third region 203 of the carbon nanotube are covered. The second region 202, by contrast, remains uncovered. Then, the hot atmosphere containing boron atoms and nitrogen atoms is generated. In the process, the carbon nanotube is only converted into a boron nitride nanotube in the uncovered second region 202. This results in the hetero-nanotube 210 illustrated in FIG. 2.

Complicated masks can be used to produce correspondingly complicated hetero-nanotubes.

The chemical substitution reaction, as an alternative to simply heating in a furnace, can be carried out by exposing the carbon nanotube which is to be converted to an atmosphere containing boron atoms and nitrogen atoms which has been moderately heated to the extent required and applying a suitable electric field to the carbon nanotube in such a manner that the chemical substitution reaction is effected, typically by catalysis by means of the electric field, so that the carbon nanotube is converted into a boron nitride nanotube.

The chemical substitution reaction in this case takes place exclusively in regions of the carbon nanotube in which the electric field has a sufficient field strength to effect the chemical substitution reaction.

The electric field is applied to the carbon nanotube in such a way that its electric field strength is only strong enough to effect the conversion in the region which is to be converted, i.e. in the example shown in FIG. 2 in the second region, and consequently the carbon nanotube is only converted into a boron nitride nanotube in the desired region which is to be converted.

The electric field used can be any desired electric field. To ensure that only the desired region (or desired regions) is converted, the electric field is shielded outside the desired region, for example by means of a suitably structured, e.g. perforated metallic foil.

Alternatively, the electric field used is the electric field of a device which generates a spatially limited electric field without the need to take further measures. By way of example, it is possible to use the elevated electric field beneath a fine tip. It is preferable to use the electric field beneath the tip of a scanning probe microscope. Beneath the tip of a scanning probe microscope, it is possible to generate an electric field whose field strength is high only in the region directly around the tip and is negligible outside this region. Consequently, the tip makes it possible for a locally delimited, very small region lying opposite the tip to be exposed to a high electric field. Therefore, if the tip is positioned at the elongate side wall of a carbon nanotube, at a suitable distance from the carbon nanotube, and a suitable electric field is applied between the tip and the nanotube, the carbon nanotube is only converted into a boron nitride nanotube in the region which lies opposite the tip.

The methods corresponding to the various embodiments can also be combined. In this case, there are regions of the hetero-nanotube whose production involves producing different nanotubes, i.e. at least one carbon nanotube and at least one boron nitride nanotube, from the outset. Moreover, there are regions of the hetero-nanotube whose production involves converting a carbon nanotube into a boron nitride nanotube.

According to one variant, in the embodiments of the method for producing a heterostructure component described above, a catalyst surface 502 which is provided at a predetermined location and is made from a catalyst material can be used during the production of any desired nanotube 501, which catalyst surface 502 causes the nanotube 501 to be produced at the predetermined location.

FIG. 5 shows a nanotube arranged on a catalyst surface, in accordance with this variant of the invention. The catalyst surface 502 allows targeted production of the nanotube 501 at the predetermined location.

1. A heterostructure component, having an individual hetero-nanotube (110), which includes:
a first region (101) made from a first nanotube material with a first value for the bandgap, and
a second region (102) made from a second nanotube material, which is different than the first nanotube material and has a second value for the bandgap, which is different from the first value for the bandgap,
the second region (102) being arranged at the upper end (103) of the first region (101) in the longitudinal direction of the hetero-nanotube (110).

2. The heterostructure component as claimed in claim 1, in which the hetero-nanotube (210) includes at least one further region (203) made from a material with a further value for the bandgap, which differs at least from the first value for the bandgap or from the second value for the bandgap, the further region (203) being arranged at the upper end (205) of the second region (202) in the longitudinal direction of the hetero-nanotube (210).

3. The heterostructure component as claimed in claim 1 or 2, in which the value for the bandgap in the first, second and further regions in each case corresponds to a conductivity characteristic from the group consisting of metallically conducting, semiconducting and insulating conductivity characteristics.

4. The heterostructure component as claimed in claim 3, in which the hetero-nanotube (110, 210) is formed as a metallically conductive carbon nanotube in at least one region in which it is metallically conducting.

5. The heterostructure component as claimed in claim 3 or 4, in which the heterostructure nanotube (110, 210) is formed as a semiconducting carbon nanotube in at least one region in which it is semiconducting.

6. The heterostructure component as claimed in one of claims 3 to 5, in which the hetero-nanotube (110, 210) is formed as an insulating carbon nanotube in at least one region in which it is insulating.

7. The heterostructure component as claimed in one of claims 3 to 6, in which the hetero-nanotube (110, 210) is formed as a boron nitride nanotube in at least one region in which it is insulating.

8. A method for producing a heterostructure component formed from a hetero-nanotube (110, 210), in which method first of all a first nanotube is produced in a first region (101, 201, 202), and then a second nanotube is produced in a second region (102, 202, 203), fitting onto the upper end (103, 204, 205) of the first nanotube in the longitudinal direction of the first nanotube, so that overall a single hetero-nanotube (110, 210) is formed from the first nanotube and the second nanotube.

9. A method for producing a heterostructure component formed from a hetero-nanotube (110, 210), in which method first of all a first nanotube is produced, then a second nanotube is produced, and then the second nanotube, fitted to the upper end (103, 204, 205) of the first nanotube in the longitudinal direction of the first nanotube, is attached to the first nanotube, so that a single hetero-nanotube (110, 210), which in a first region (101, 201, 202) comprises the first nanotube and in a second region (102, 202, 203) comprises the second nanotube, is formed from the first nanotube and the second nanotube.

10. The method as claimed in claim 8 or 9, in which a process selected from the group of processes consisting of vapor phase epitaxy, arc discharge techniques and laser ablation, is used to produce the first nanotube and/or the second nanotube.

11. The method as claimed in one of claims 8 to 10, in which a catalyst surface (502) made from a catalyst material, which is provided at a predetermined location, is used during the production of at least one nanotube (501) of the nanotubes, which catalyst surface (502) causes the nanotube (501) to be produced at the predetermined location.

12. A method for producing a heterostructure component formed from a hetero-nanotube (110, 210), in which method first of all a carbon nanotube is produced, and then the carbon nanotube is converted into a boron nitride nanotube in at least a second partial section.

13. The method as claimed in claim 12, in which the carbon nanotube is converted into a boron nitride nanotube as a result of a chemical substitution reaction being carried out.

14. The method as claimed in claim 13, in which when the chemical substitution reaction is being carried out the first partial section is masked in such a way that it is shielded from the chemical substitution reaction, so that the chemical substitution reaction takes place only in the second partial section.

15. The method as claimed in claim 13 or 14, in which a suitable electric field is applied to the carbon nanotube in such a manner that the chemical substitution reaction is effected, so that the carbon nanotube is converted into a boron nitride nanotube.

16. The method as claimed in claim 15, in which the electric field used is the electric field beneath the tip of a scanning probe microscope.

17. The heterostructure component as claimed in claim 2, in which the first region is formed from a first metallically conducting carbon nanotube (201), the second region is formed from an insulating boron nitride nanotube (202), and the further region is formed from a metallically conducting carbon nanotube (210), the second region being formed as a tunnel junction between the first and third regions.
